(19) United States
(12) Patent Application Publication
(10) Pub. No.: US 2012/0323205 A1
(43) Pub. Date: Dec. 20, 2012

(54) Burn diaper

(76) Inventors: Sarah Beth Vanderwagen, Columbia, MD (US); Nicole Garra Cohen, Washington, DC (US)

(21) Appl. No.: 13/134,738

(22) Filed: Jun. 15, 2011

(51) Int. Cl. A61L 15/44

(52) U.S. Cl. 604/385.06; 53/111 R

(57) ABSTRACT

An embodiment includes a sterile package containing a disposable diaper with a dressing attached to the inner surface of the diaper. The dressing is impregnated with an antibiotic or other therapeutic ointment containing bacitracin. Embodiments are used as dressings for pediatric burn patients. Embodiments allow the replacement of dressings in such patients by a single care-giver. Other embodiments are used with adult patients with wounds in the pelvic area.

### BACKGROUND OF THE INVENTION

1. Field of the Invention

Embodiments include a sterile combination diaper and dressing.

2. Description of Related Art

including information disclosed under 37 CFR 1.97 and 1.98.

Extensive burns about the pelvic region of infants and children often result from bathing the child in excessively hot water and from other hot liquids spilled on small children. Such burns present major problems to a pediatric burn ward, as they often become infected and are difficult to manage due to the periodic contamination of the wounds from the child's urine and stool. Current treatment involves cleaning the wounds, wrapping the wound area with a methylcellulose dressing impregnated with antibiotic or other therapeutic ointment, and covering the dressing with a disposable diaper. This procedure involves two skilled professionals in most cases and often must be repeated several times a day. Current procedures are time-consuming and result in considerable discomfort for the patients.

A current treatment for second to third degree burns upon initial presentation to the burn center is to first debride the dead tissue, then apply an ointment containing an effective amount of the antibiotic to a methylcellulose dressing, which is placed on the burned skin. When the area of skin that is burned is on an extremity or the trunk, the methylcellulose dressing is then covered with a sterile gauze dressing and secured with tape or an elastic fiber based dressing. For those burns on the trunk or an extremity, a sterile dressing change is performed once daily and reinforced as needed if the dressing is soiled. Once daily dressing changes are done in a treatment room separate from the patient's room help to minimize anxiety and emotional trauma to the child.

Treatment of burns to the lower abdomen, perineum, and buttock region also involve an initial debridement of the dead tissue, application of antibiotic or other therapeutic ointment, and coverage with a methylcellulose dressing. For young, incontinent children or children who developmentally regress following the trauma of a major burn, a disposable diaper is then applied over the methylcellulose dressing. This is problematic for the patient and the nursing staff of the burn center in several ways.

When a burn is on an extremity or the trunk the methylcellulose dressing is secured reasonably well by the external roll dressing, ensuring that during patient movement the antibiotic or other therapeutic ointment and methylcellulose dressing stays in contact with the burned skin. While the antibiotic or other therapeutic ointment promotes healing by preventing infection and keeping the skin moist, the methylcellulose dressing keeps the antibiotic or other therapeutic ointment in place and acts as a physical barrier to protect the burned skin and to prevent friction between the burned skin and the outer portion of the dressing.

When a burn is under the diaper area and the methylcellulose dressing is not held in place by anything other than the diaper, the dressing tends to slide off of the burned skin when the patient moves. This leads to increased pain for the patient because of friction between the burned skin and the absorbent surface of the diaper. Also, if the diaper absorbs the antibiotic or other therapeutic ointment the burn wound can become dry, which impedes healing. Encouraging patient movement is imperative to promote skin elasticity during the healing process and prevent contracture formation. The burn diaper of the present disclosure has a methylcellulose dressing as the top layer of the absorbent surface of the diaper in every area that comes in contact with the patient's skin. The patient can move without additional pain caused by friction because the antibiotic or other therapeutic ointment and the methylcellulose dressing stays in place. In addition, the areas of the methylcellulose dressing which is not coated with ointment remains permeable to liquids, allowing the diaper to absorb urine.

Typically, each time the patient with a burn under the diaper area urinates or has a bowel movement a sterile dressing change must be done. Currently this procedure requires two nurses, one to remove the diaper and cleanse the skin, while the second nurse, using sterile technique, applies antibiotic or other therapeutic ointment to strips of methylcellulose dressing cut to match the size of the patient's burn area. A clean disposable diaper is then placed on the patient. Often burn patients have frequent loose stools as side effects of necessary antibiotics and other medications. It is essential to remove stool and urine from burned skin as soon as possible because both substances are caustic to burned skin and retard healing.

Since two nurses are required each time a burned child's diaper must be changed, this process is expensive, can take away from the quality of care other patients on the unit receive, and is a significant drain on hospital resources. Additionally, frequent lengthy dressing changes can lead to increased pain and anxiety and reduced time for rest for the patient.

The burn diapers of this disclosure will streamline the diaper and dressing change process. The burn diaper comes in a sterile package. One nurse can simply first use sterile technique to prepare the burn diaper by applying antibiotic or other therapeutic ointment to the appropriate parts of the burn diaper, then quickly remove the soiled diaper from the patient, cleanse the skin, and apply the already prepared burn diaper. Urine is free to pass through the areas of the dressing which are free of ointment where the urine is absorbed as with conventional diapers. This reduces the frequency of diaper changes. Use of the burn diapers of the present disclosure with burn patients shortens the process of changing diapers, decreases patient discomfort, frees up nurses' time and reduces costs. Furthermore, a simplified dressing change process allows the family of the patient to care for the burn wound at home more easily, which can shorten the length and frequency of hospital admissions, further reducing costs.

The burn diapers of the present disclosure also solves problems associated with burns located on the upper thigh and hips. Burns in such areas present challenges because of the difficulty in wrapping the burned area with dressings, as would be done with burned areas on the extremities. The burn diaper of the present disclosure covers more area of the hips and upper thighs than a standard diaper, allowing continuous friction-free contact between burned skin and methylcellulose dressing coated with antibiotic or other therapeutic ointment.

Embodiments of the present disclosure include a disposable package containing a sterile dressing-diaper combination in which the dressing or portions of the dressing may optionally be impregnated with ointment containing an antibiotic.

U.S. Pat. No. 4,341,208 discloses a moisture-retaining covering for ointment.

U.S. Pat. No. 4,560,381 discloses a disposable panty with a removable feminine napkin.

U.S. Pat. No. 4,620,849 discloses a cover for small animals which applies pharmaceutical compositions to the animals.

U.S. Pat. No. 5,643,588 discloses a diaper having a topsheet which passes urine and is coated with a lotion composition.

U.S. Pat. No. 6,979,325 discloses a post-circumcision diaper with a plastic protective cup used to protect a baby's penis against direct and incidental pressure.

U.S. Pat. No. 7,033,645 discloses a diaper with a stable skin care composition on its skin-contacting surface.

Pub. U.S. Pat. Applic. 2008/0195065 discloses a pad which may be placed in a diaper which contains dried milk or frozen milk and is used to treat skin irritations such as diaper rash.

Pub. U.S. Pat. Applic. 2009/0062753 discloses a women's panties or men's brief lined with local anesthetics to create an area of numbness after surgery or other painful procedure, such as waxing for hair removal.

None of the discovered prior art includes the advantages of embodiments of the present disclosure, some of which are: providing a sterile dressing-diaper combination, or burn diaper which allows changing the diaper by a single care-giver, and which allows quicker dressing replacement with less manipulation, thereby minimizing discomfort to the patient.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY OF THE INVENTION.

Embodiments include a combination diaper dressing system for treatment of pediatric burn victims which comprises a diaper comprising an outer impervious layer and an inner absorbent layer. Attached to the diaper is a dressing, the dressing covering the inner absorbent layer. In use, the portion of the dressing which comes into contact with the patient's burned area is coated by the care-giver with an ointment which may contain antibiotic, such as bacitracin. In embodiments a portion of the dressing is coated with an antibiotic-containing ointment before packaging. The diaper and dressing are enclosed within a wrap and the diaper, dressing, and inner surface of the wrap are sterile.

The following embodiments and aspects thereof are described and illustrated in conjunction with systems, tool and methods which are meant to be exemplary and illustrative, not limiting in scope. In various embodiments, one or more of the above—described problems have been reduced or eliminated, while other embodiments are directed to other improvements.

In addition to the exemplary aspects and embodiments described above, further aspects and embodiments will become apparent by reference to the drawings and by study of the following descriptions.

### BRIEF DESCRIPTION OF THE SEVERAL VIEWS OF THE DRAWING(S).

FIG. 1 is a view of the patient contact surface of a first embodiment burn diaper.

FIG. 2 is a cross-sectional view of a first embodiment burn diaper taken at line 2-2 of FIG. 1.

FIG. 3. is a view of the patient contact surface of a second embodiment burn diaper.

FIG. 4 is a cross-sectional view of a second embodiment burn diaper taken at line 4-4 of FIG. 3.

FIG. 5 is a view of a first embodiment burn diaper folded and enclosed within a sterile package.

FIG. 6 is a cross-sectional view of a first embodiment burn diaper folded and enclosed within a sterile package taken at line 6-6 of FIG. 5.

FIG. 7 is a view of a third embodiment burn diaper enclosed is a sterile package.

FIG. 8 is a cross-sectional view of a third embodiment burn diaper enclosed within a sterile package taken at line 8-8 of FIG. 7.

FIG. 9 is a front view of a third embodiment burn diaper outside of a package.

### DETAILED DESCRIPTION OF THE INVENTION

In this disclosure the term “therapeutic ointment” means a salve or unguent which contains a therapeutic ingredient, such as a antibiotic, antimicrobial, or antibody. The term “bacitracin” means an ointment containing an effective amount of the antibiotic bacitracin. The term “dressing” means a flexible, permeable, open celled or open woven fabric covering placed in contact with a wound or burn and which serves to absorb exudate, protect from infection, and promote healing. The term “therapeutic ointment” means a salve or unguent which contains therapeutic ingredients such as antibiotics, antimicrobials, or antibodies.

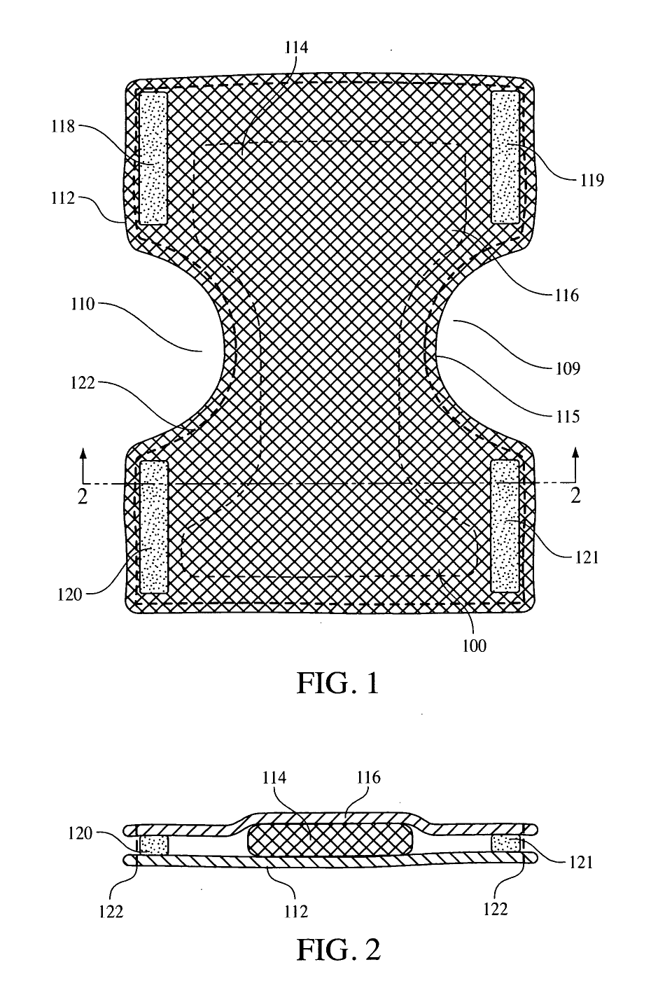

FIG. 1 is a view of the patient contact surface of a first embodiment burn diaper 100. Visible in FIG. 1 is an approximately hourglass-shaped dressing 116 which receives the therapeutic ointment. The dressing covers the entire inner surface of the burn diaper. The dressing is permeable to urine in areas which do not receive the therapeutic ointment. Below the dressing 116 layer and outlined in FIG. 1 by a dashed line, is a hourglass-shaped absorbent layer 114 which absorbs and retains urine. Below the absorbent material 114 is the hourglass-shaped outer layer 112 of the burn diaper. The dressing 116 is attached to the outer layer 112 by stitching 122 around the perimeter of the wound diaper. Alternatively, other means of attaching the dressing and outer layer, such as adhesive, heat welding, may be used. Hook and loop attachment strip are arrayed near the ends of the wound diaper and are used to retain the diaper in place on a patient. A left hook strip 118 is attached to the dressing at the upper left corner. It interacts with a left loop strip 120 attached to the lower left corner of the wound diaper in order to secure the burn diaper on the patient. A right hook strip 119 is attached to the dressing at the upper right corner. It interacts with a right loop strip 121 attached to the lower right corner of the wound diaper. Both the hook and loop areas are on the patient side of the burn diaper, are covered by the dressing, and are able to interact by protrusion through the dressing. A left leg cut out 109 and right leg cut out 110 are shown. The approximate middle of the length of the wound diaper is indicated at 115.

FIG. 2 is a cross-sectional view of a first embodiment burn diaper taken at line 2-2 of FIG. 1. Visible in FIG. 2 is the outer layer 112, the absorbent layer 114, and dressing 116. Stitching 122 connects the dressing 116 and outer layer 112. The left loop strip 120 and right loop strip 121 are also shown in FIG. 2.

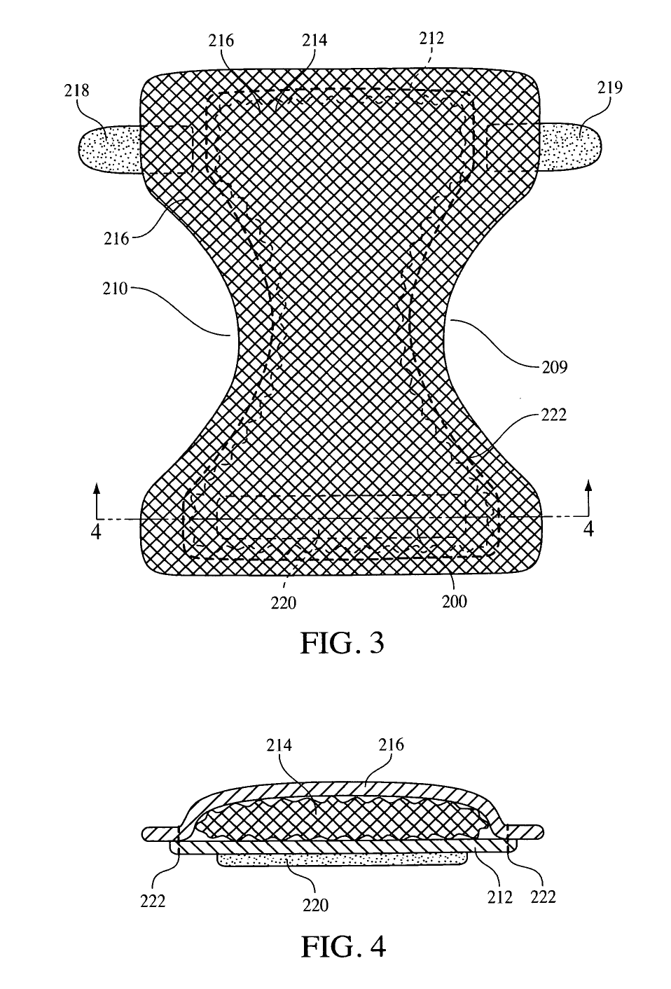

FIG. 3. is a view of the patient contact surface of a second embodiment 200 burn diaper. The second embodiment differs from the first embodiment in the location of hook and loop connectors which connect the front and back portions of the burn diaper about the patient's torso. In the first embodiment burn diaper the hook connectors are on strips approximately parallel to the sides at one end of the burn diaper and the loop connectors are on strips approximately parallel to the sides at the other end of the burn diaper. In the second embodiment the hook connectors are on tabs which extend from the sides at one end of the burn diaper and the loop connectors are on a band approximately parallel to the other end of the burn diaper.

Visible in FIG. 3 is an approximately hourglass-shaped dressing 216 which receives the therapeutic ointment. The dressing is permeable to urine in areas which do not receive the therapeutic ointment. Below the dressing 216 layer and outlined in FIG. 3 by a dashed line, is a hourglass-shaped absorbent layer 214 which absorbs and retains urine. Below the absorbent material 214 is the hourglass-shaped outer layer 212 of the burn diaper. The absorbent material 214 is attached by adhesive to the outer layer 212. The dressing 216 is attached to the outer layer 212 by stitching 222 or other suitable adhesive means around the perimeter of the wound diaper Also visible in FIG. 3 is the left hook area 218 on a tab on the left side of the burn diaper and right hook area 219 on a tab on the right side of the burn diaper, as well as the loop area 220, which is approximately parallel to the end of the burn diaper. The hook 218 and 219 tabs are on the patient side and the loop area 220 is on the outer layer other side of the burn diaper. A left leg cut out 209 and right leg cut out 210 are shown. Stitching 222 which fastens the dressing to the outer layer also is shown by a dotted line in FIG. 3.

FIG. 4 is a cross-sectional view of a second embodiment burn diaper taken at line 4-4 of FIG. 3. Visible in FIG. 4 is the outer layer 212, the absorbent layer 214, the dressing 216, and the loop area 221 of the hook and loop fastener. The stitching 222 which connects the dressing to the outer layer is shown.

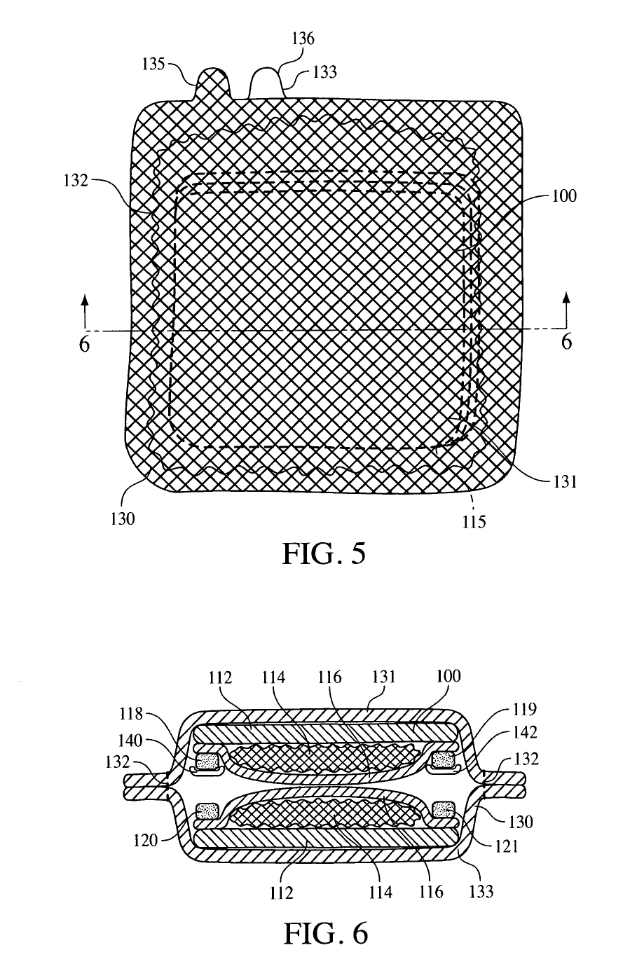

FIG. 5 is a view of a sterile first embodiment burn diaper folded and enclosed within a sterile package. The first embodiment burn diaper is folded along the middle (115 in FIG. 1) with the dressing 116 in contact with itself. In FIG. 5 the burn diaper 100 is contained in a sterile covering or package 130 with the fold 115 at the bottom of the package. The covering 130 is comprised of a front panel 131 and a back panel (133, visible only as tab 136 in FIG. 5) and is sealed about the perimeter by fusion of the front 131 and back 133 panels in a fused area about the perimeter of the package. A front 135 tear tab on the front panel and a back 136 tear tab on the back panel are located at the top of the package for easy opening of the sterile package. To open the package the user grasps the front tear tab 135 and the back tear tab 136 and pulls the panels apart, thereby exposing the sterile field containing the sterile burn diaper.

FIG. 6 is a cross-sectional view of a sterile first embodiment burn diaper folded and enclosed within a sterile package taken at line 6-6 of FIG. 5. Visible in FIG. 6 is the front panel 131 and back panel 133 of the covering 130 and the fused area 132 which connects the front and back panels. Elements of the folded burn diaper 100 visible in FIG. 6 include the outer layer 112, absorbent layer 114 and dressing 116. Also visible are the left hook 118 area and the right hook 119 area of the hook and loop fastener, as well as the left loop 120 and right loop 121 areas. Left 140 and right 142 release sheets are imposed between the hook and loop areas to prevent fastening between the hook and the loop areas while the burn diaper is enclosed in the package. In use, the release sheets are removed and discarded after the package is opened.

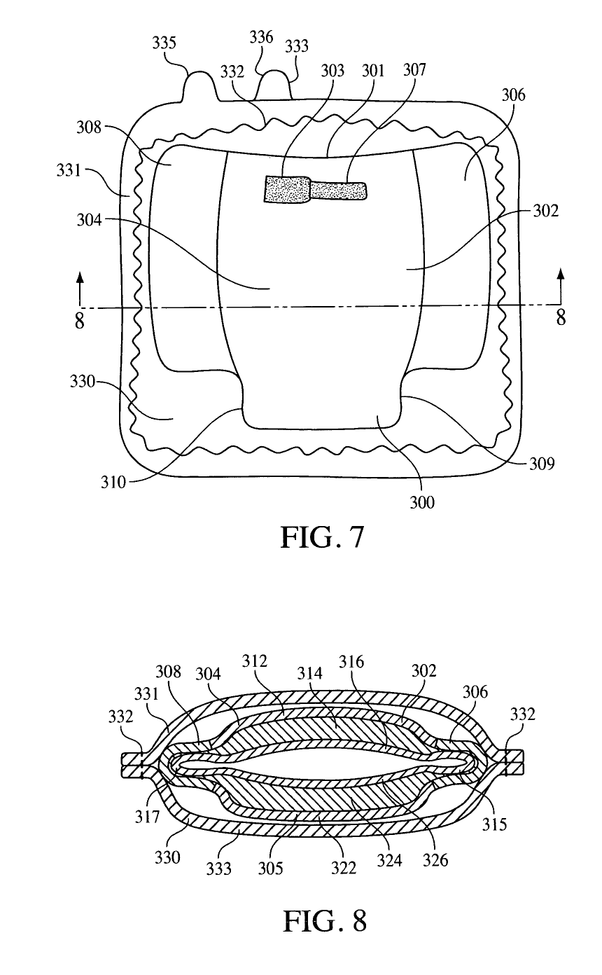

FIG. 7 is a view of a sterile third embodiment pull up 300 burn diaper enclosed is a sterile package. The third embodiment differs from the first and second embodiments in that it has the form of a jockey brief, also called a support brief, or male swim suit type. This embodiment has an open top and two leg holes and is placed on the patient by putting the feet into the open top and through the leg holes. In the third embodiment the dressing covers the entire interior of the burn diaper including the elastic connector panels along the sides.

Also visible in FIG. 7 is the third embodiment brief-type burn diaper or pull up diaper 302 comprised of a front panel 304 attached to a left elastic connector panel 306 and to a right elastic connector panel 308. The right leg hole 310 and left leg hole 309 are indicated in FIG. 7. The open top 301 is shown, as well as a hook and loop tab 303 which interacts with a hook and loop strip 307 and serves to tighten the burn diaper about the wearer's waist. Other means of retaining the burn diaper, such as embedded elastic material about the top, may be used. Also visible in FIG. 7 is the sterile package 330 comprised of a front panel 331 and a back panel (333, visible only as tab 336 in FIG. 7), with the front and back panels of the package sealed together by a sealing area 332. A front tear tab 335 and a back tear tab 336 are located at the top of the package for easy opening of the sterile package. To open the package the user grasps the front tear tab 335 and back tear tab 336 and pulls the panels apart, thereby exposing the sterile field containing the sterile burn diaper.

FIG. 8 is a cross-sectional view of a third embodiment burn diaper enclosed within a sterile package taken at line 8-8 of FIG. 7. Visible in FIG. 8 is the package 330 comprised of front panel 331, back panel 333, and sealing area 332 which connects the panels about the periphery of the package and maintains sterility of the package. Elements of the third embodiment burn diaper 302 visible in FIG. 8 include the front panel 304, front panel outer layer 312, front panel absorbent layer 314, and front panel dressing 316. Also visible is the left elastic connector area 306 which connects the front 304 and back 305 panels on the left side of the burn diaper, as well as the right elastic area 308 which connects the front 304 and back 305 panels on the right side of the burn diaper. Finally, visible in FIG. 8 is the back panel 305, back panel outer layer 322, back panel absorbent layer 324, and back panel dressing 326. FIG. 8 also shows the portion 317 of the dressing which covers the right elastic area 308, and the portion 315 which covers the left elastic area 306. This embodiment insures that all portions of the inner surface of the wound diaper are covered with dressing.

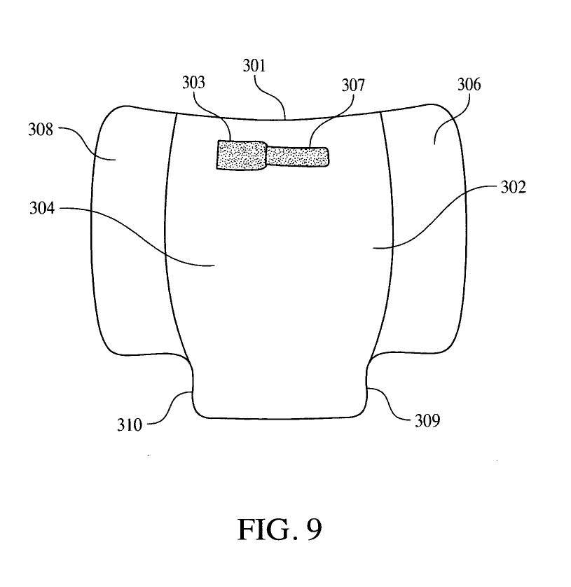

FIG. 9 shows the third embodiment brief-type burn diaper 302 outside a package. The third embodiment is comprised of a front panel 304 attached to a left elastic connector panel 306 and to a right elastic connector panel 308. The right leg hole 310 and left leg hole 309 are indicated in FIG. 9. The open top 301 is shown, as well as a hook and loop tab 303 which interacts with a hook and loop strip 307 and serves to tighten the burn diaper about the wearer's waist. Other means of retaining the burn diaper, such as embedded elastic material about the top, may be used.

In all embodiment burn diapers dressing covers the entire inner surface of the diaper adjacent to the skin of the patient. This insures the contact of antibiotic or other therapeutic ointment with the burns or other wounds wherever they may occur on the pelvic and upper thigh areas.

The first two embodiment wound diapers are particularly useful for pediatric patients with extensive burns in the pelvic area because of the ease of application to the patient. The hook and loop arrangement allows application of the diaper with minimal pain to a patient with burns on the lower abdomen and upper thighs. Use of the third embodiment diaper with such patients might cause pain from the need to place the patient's legs through the leg holes, and the friction from moving the diaper into place. The third embodiment is best used for patients free of burns or injury in the upper thigh and crotch area.

In embodiments the dressing is manufactured of any open or loosely woven, soft, non-toxic fabric dressing which is nonadherent to wounds. Suitable materials include methylcellulose, linen, and cotton. In embodiments the dressing is fastened to the outer layer by stitching with thread. Other suitable methods of attaching the dressing include fusing and adhesives. A suitable dressing is the CURITY methylcellulose non-adhering dressing, Ref. 6115. CURITY is a trademark for surgical and medical disposable drapes, sheets, covers, towels, leggings, and pads owned by Tyco Healthcare Group LP. Dressings are obtainable from Tyco Healthcare Group LP, Mansfield, Mass. Other suitable dressings include ADAPTIC methylcellulose dressings obtainable from Systagenix Wound Management (US), Inc., New York, N.Y. ADAPTIC is a trademark for non-adhering dressings for wounds owned by Systagenix Wound Management (US), Inc.

The outer layer is manufactured of any pliable, strong, material. Such materials, such as cellulose, are conventionally used as the outer layer (layer not in contact with the patient or wearer) of conventional diapers. The outer layer may optionally be of water or fluid impermeable material. Suitable materials include plastics such as polyethylene and polyvinyl chloride,

The absorbent layer is manufactures of flexible, non-irritable absorbent material capable of absorbing or holding urine, feces, or other bodily exudates, such as materials used in conventional disposable diapers. Suitable materials include wood pulp, cellulose, synthetic fibers and polymers. The absorbent layer optionally has a woven layer or cover or topsheet on the patient side of the absorbent layer. Elastic materials optionally may be incorporated along the sides of the absorbent layer to help prevent leakage from the wound diaper about the patient's legs.

In embodiments any suitable flexible, strong, material impervious to microbes and suitable for sterilization may be used for the package. Suitable materials include flexible plastic such as polyethylene, polyvinyl chloride, or ethylene polyfluoroethylene, paper, and cotton fabric. Optionally, sterile assembly may be used in which the wound diaper itself is sterilized and then packaged under sterile conditions in a sterile package.

In embodiments the bacitracin or other therapeutic substances is applied by the care provider under sterile conditions to the portion of the burn diaper that will come into contact with the burned skin just before the burn diaper is applied to the patient. This minimizes needless contact of the therapeutic ointment with unburned skin and also allows areas of the dressing not covered with ointment to be permeable to urine.

Other embodiments are specifically contemplated in which the dressing is impregnated with antibiotics or other therapeutic substances before the burn diaper is packaged and sterilized. Embodiments in which only a specific portion of the dressing is impregnated before packaging and sterilization are specifically contemplated.

Although the examples described in this disclosure are used with burn wounds, it is specifically contemplated that other types of wounds may be treated using a wound diaper. Although the examples described in this disclosure are used with infants, it is specifically contemplated that embodiments may be used with patients of any age.

In embodiments, any conventional method of sterilization which kills microorganisms and viruses and is compatible with the materials of manufacture may be used. These include such methods as steam sterilization, dry heat sterilization, microwave sterilization, exposure to dry heat, hydrogen peroxide, ozone, ethylene oxide or ionizing radiation.

In embodiments the dressing covers the upper thighs of the patient when the burn diaper is applied. This allows treatment of bums and wounds on the upper thighs.

In use, the care provider obtains a sterile packaged burn diaper which is not treated with therapeutic ointment, then using sterile procedures, opens the package, unfolds the folded diaper, removes and discards the release sheets if present, adds sterile bacitracin or other therapeutic ointment to the appropriate region of the dressing, applies the burn diaper to the patient, and, if appropriate, closes the fasteners which retain the burn diaper on the patient.

The process of treating a pediatric burn patient with burns in the pelvic area in need of a dressing change comprises the steps:
a. obtaining a burn diaper comprising an outer layer and an inner absorbent layer, a dressing attached to the diaper, the dressing covering the inner absorbent layer, fasteners used to secure the burn diaper on a patient, and a package enclosing the diaper and dressing, wherein the diaper, dressing and inner surface of the package are sterile,
b. removing the burn diaper from the package,
c. applying therapeutic ointment to a portion of the dressing corresponding to the burned area on the patient,
d. removing the soiled diaper from the patient,
e. cleaning the patient of inflammatory products, urine and stool,
f. applying the diaper with attached dressing to the pelvic region of the patient, and
g. securing the burn diaper on the patient using the fastenings on the diaper.

If the burn diaper is one which already has therapeutic ointment applied to the dressing, step c. is omitted.

Although only the process of tearing open the package by hand at the tear area is disclosed, other methods of opening the sterile package are contemplated, such as cutting the package with a scissors or scalpel.

While a number of exemplary aspects and embodiments have been discussed above, those of skill in the art will recognize certain modifications, permutations, additions and sub combinations thereof. It is therefore intended that the following appended claims and claims hereafter introduced are interpreted to include all such modifications, permutations, additions and sub-combinations as are within their true spirit and scope. The applicant or applicants have attempted to disclose all the embodiments of the invention that could be reasonably foreseen. There may be unforeseeable insubstantial modifications that remain as equivalents.

I claim:

1. A burn diaper for treatment of pediatric burn victims comprising:
a diaper approximately rectangular in shape having long sides and short sides,
the diaper comprising an outer impervious layer and an inner absorbent layer,
an approximately rectangular dressing attached to the diaper, the dressing covering the inner absorbent layer,
fasteners capable of reversible interaction with each other attached to the dressing and the diaper at the ends of the long sides, and
a package enclosing the diaper and dressing, wherein
the diaper, dressing and inner surface of the package are sterile.

2. The burn diaper of claim 1 further comprising:
a therapeutic ointment applied to the dressing.

3. The burn diaper of claim 1 further comprising:
an ointment containing bacitracin applied to the dressing.

4. The burn diaper of claim 3 wherein the bacitracin is selected from the group consisting of bacitracin A, bacitracin methylenedisalicylic acid, and zinc bacitracin.

5. The burn diaper of claim 3 wherein the bacitracin is zinc bacitracin.

6. The burn diaper of claim 1 wherein the fasteners are hook and loop fasteners.

7. The burn diaper of claim 1 wherein the diaper, dressing and inner surface of the package are sterilized by a method selected from the group consisting of steam sterilization, dry heat sterilization, microwave sterilization, exposure to dry heat, exposure to hydrogen peroxide, exposure to ozone, exposure to ethylene oxide and exposure to ionizing radiation.

8. The combination diaper dressing system of claim 1 wherein the diaper, dressing and inner surface of the package are sterilized by ionizing radiation.

9. The burn diaper of claim 1 wherein the diaper, dressing, and packaging are sterilized separately and the diaper and dressing and the assembled within the package by sterile assembly.

10. A burn diaper for treatment of pediatric burn victims comprising:
a diaper approximately hour-glass in shape,
the diaper comprising an outer impervious layer and an inner absorbent layer,
an approximately rectangular dressing attached to the diaper, the dressing covering the inner absorbent layer,
the dressing extending beyond the sides of the diaper,
fasteners capable of reversible interaction with each other attached to the outer side of the impervious layer, and
a package enclosing the diaper and dressing, wherein
the diaper, dressing and inner surface of the package are sterile.

11. The burn diaper of claim 10 further comprising:
a therapeutic ointment applied to the dressing.

12. The burn diaper of claim 10 further comprising:
an ointment containing bacitracin applied to the dressing.

13. The burn diaper of claim 12 wherein the bacitracin is selected from the group consisting of bacitracin A, bacitracin methylenedisalicylic acid, and zinc bacitracin.

14. The burn diaper of claim 12 wherein the bacitracin is zinc bacitracin.

15. The burn diaper of claim 10 wherein the fasteners are hook and loop fasteners.

16. The burn diaper of claim 10 wherein the diaper, dressing and inner surface of the package are sterilized by a method selected from the group consisting of steam sterilization, dry heat sterilization, microwave sterilization, exposure to dry heat, exposure to hydrogen peroxide, exposure to ozone, exposure to ethylene oxide and exposure to ionizing radiation.

17. The combination diaper dressing system of claim 10 wherein the diaper, dressing and inner surface of the package are sterilized by ionizing radiation.

18. The burn diaper of claim 10 wherein the diaper, dressing, and packaging are sterilized separately and the diaper and dressing and the assembled within the package by sterile assembly.

19. The process of treating a pediatric patient with burns in the pelvic region comprising the steps:
a. obtaining a burn diaper comprising an outer layer and an inner absorbent layer, a dressing attached to the diaper, the dressing covering the inner absorbent layer, fasteners used to secure the burn diaper on a patient, and a package enclosing the diaper and dressing, wherein the diaper, dressing and inner surface of the package are sterile,
b. removing the burn diaper from the package,
c. applying therapeutic ointment to a portion of the dressing corresponding to the burned area on the patient,
d. removing the soiled diaper from the patient,
e. cleaning the patient of inflammatory products, urine and stool,
f. applying the diaper with attached dressing to the pelvic region of the patient, and
g. securing the burn diaper on the patient using the fastenings on the diaper.
